(19) United States
(12) Patent Application Publication
(10) Pub. No.: US 2018/0094236 A1
(43) Pub. Date: Apr. 5, 2018

(54) CELL LINES THAT ARE FREE OF VIRAL INFECTION AND METHODS FOR THEIR PRODUCTION

(71) Applicant: Boyce Thompson Institute for Plant Research Inc., Ithaca, NY (US)

(72) Inventors: Robert Granados, Ithaca, NY (US); Gary Blissard, Ithaca, NY (US); Paul Debbie, Ithaca, NY (US)

(21) Appl. No.: 15/560,656

(22) PCT Filed: Mar. 23, 2016
(86) PCT No.: PCT/US2016/023816

§ 371 Date: Sep. 22, 2017

Related U.S. Application Data
(60) Provisional application No. 62/136,880, filed on Mar. 23, 2015.

(51) Int. Cl. C12N 5/07; C12N 5/00

(52) U.S. Cl. CPC C12N 5/06; C12N 5/0081; C12N 2770/30021; C12N 2760/20021; C12N 5/0018

(57) ABSTRACT

The present invention relates to cells and cell lines that are free of viral contamination and methods for eliminating viral contamination from a cell or cell line. One exemplary method developed generates Trichoplusia ni cell lines that are free of alphanodavirus. Methods of using a specific, virally-infected cell to generate a virus-free cell are also described herein.

### CROSS-REFERENCE TO RELATED APPLICATIONS

This application claims the benefit of the filing date of U.S. Provisional Application No. 62/136,880, filed Mar. 23, 2015. The entire content of that earlier-filed application is hereby incorporated by reference herein.

### FIELD OF THE INVENTION

The present invention relates to cells and cell lines that are free of viral contamination, and more particularly to Trichoplusia ni cells that are free of alphanodavirus. Methods of making a virus-free cell, cell culture, or cell line are also described herein.

### BACKGROUND

Trichoplusia ni, more commonly referred to as the cabbage looper, is a moth indigenous to many regions of the world, including North America, parts of Europe and Africa, and much of Asia. Trichoplusia ni is of interest and concern to the agricultural industry, and it is also the source of a cell line that has been extensively used as a tool by molecular biologists. For example, GlaxoSmith Kline used the HighFive™ cell line in the production of their FDA approved bivalent Human Papilloma Virus (HPV) vaccine (Rebeaud and Bachman, Innovation in Vaccinology: From Design, Through to Delivery and Testing, Springer Science, Jul. 20, 2012, page 106).

### SUMMARY

The present invention is based, in part, on our discovery of a method for the production of an improved version of the BTI-TN-5B1-4 cell line, which constitutes the parental cell line known commercially as the High Five™ cell line (BTI-TN-5B1-4; Life Technologies, Invitrogen, Carlsbad, Calif.). The present cells are improved in that they are free of the alphanodavirus carried by the parental cell line. Although there is likely to be variability depending on the construct used and other factors, the alphanodavirus-free High Five™ cells can generate high levels (e.g., experimentally and commercially useful amounts) of recombinant proteins when used with a baculovirus expression vector. Also, and relative to the parental cell line, purification of recombinant proteins or products such as Virus Like Particles (VLPs) from an alphanodavirus-free cell line is less difficult because there is no need to remove alphanodavirus particles during the purification process or to monitor for the absence of the virus in final, purified products made by the virus-free cells. This makes the present cells easier to use, particularly when making therapeutic proteins for administration to humans.

Accordingly, in a first aspect, the invention features methods of making a virus-free cell or a cell line from a virus-infected cell or cell line. We use the terms “virus-free,” “free of virus(es),” “not infected with a/the virus,” and the like to indicate that a virus in question (e.g., an alphanodavirus) is absent from a given cell or cell line insofar as one can determine using currently available detection methods (i.e., that the virus, if present at all, is present below the level at which it can be detected). While generating virus-free cells or cell lines is likely to be preferable in most instances, the methods of the invention can also be used to generate a cell, cells and/or cell lines in which the viral load has been reduced (e.g., by at least or about 25%, 50%, 75%, 80%, 85%, 90%, 95%, 99%, or values therebetween, at the conclusion of the method). Where a cell is infected with more than one type of virus, the methods of the invention may be used to eliminate or reduce the load of just one of the types of virus present, more than one of the types, or all of the types. The methods, whether employed to eliminate one or more viruses from a cell or to reduce the viral load, can be carried out by co-culturing a first cell that is infected with the virus and a second cell that is not infected with the virus or not susceptible to infection with the virus. The cells are maintained in culture until the virus is undetectable in the first cell or reduced to a desired level or by a desired amount. In another aspect, the invention features methods of making virus-free cells or reducing viral load by employing a culture medium (or a fraction thereof) obtained from a culture of cells that are virus-free and/or not susceptible to viral infection. For example, one can culture a first cell that is virus-infected with culture medium (or a fraction thereof) in which a second cell that is virus-free has been cultured.

In either aspect, the virus can be one within the family Ascoviridae, Baculoviridae, Birnaviridae, Dicistroviridae, Iridoviridae, Metaviridae, Nodaviridae, Parvoviridae, Polydnaviridae, Poxviridae, Pseudoviridae, Rhabdoviridae, Reoviridae, or Tetraviridae. Further, the virus within any of these families can be an RNA virus and can, even further, be a single stranded RNA (ssRNA) virus (e.g., a positive sense (+), negative sense (−), or antisense ssRNA virus). For example, the ssRNA virus can be a negative sense (−) ssRNA virus with the family Rhabdoviridae. Where the virus is within the family Rhabdoviridae, it can further be within the genus Cytorhabdovirus. In some embodiments, the (−) ssRNA virus is Sf-rhabdovirus. In other embodiments, where the virus is within the genus Alphanodavirus, the virus can be Tn5 cell line virus (TNCLV) (see Li et al., J. Virol. 81:10890-10896, 2007), Nodamura Virus, Flock House Virus (FHV), Black Beetle Virus, Boolarra Virus, or Pariacoto Virus. Additional types of viruses that can be reduced or eradicated by the present methods are described below.

The co-culture can be carried out in various ways, several of which are exemplified in the Examples provided below. For example, in one embodiment, a co-culture is generated by placing the first and second cells in the same tissue culture vessel (e.g., a plate, tube, or flask) without any barrier to impede contact between either the first and second cells or the medium in which they are cultured. In another embodiment, one can place the first and second cells in the same tissue culture vessel with a barrier that impedes contact between the first and second cells but does not impede transfer of the medium and any compounds smaller than the pore size of the barrier in which they are grown. Thus, the first and second cells can be exposed to the same tissue culture medium and/or any agents secreted by the first or second cells.

The first and second cells can be of the same type. For example, the first and second cells can be of the same species and genus and may be clonally related. However, one cell (e.g., the first cell) can be infected with a virus while the other (e.g., the second cell) is not. The first and second cells can also be different from one another. For example, the first cell and the second cell can be different species within a genus, they can be of different genera, or they may have been genetically modified in different ways. The first and/or second cell can be an insect cell. In one embodiment, the first cell can be of a cell line derived from Trichoplusia ni (e.g., of the cell line BTI-TN-5B1-4, H5CL-B (ATCC Accession No. PTA-5635), HSCL-F (ATCC Accession No. PTA-5636), BTI-TN-MG1 (ATCC Accession No. CRL-10860), or Hink's Trichoplusia ni (TN-368) cell line. The second cell can be of a primary culture of Manduca sexta cells; of a Manduca sexta cell line; or of the Sf9 cell line (an insect cell line derived from the parental Spodoptera frugiperda cell line IPLB-Sf-21-AE). In some embodiments, the second, virus-free cell is cultured alone prior to the addition of the first cell. For example, the second cell can be cultured until colonies form prior to the addition of the first cell. As a result, the culture can include a limited number of the first cell, and there can be, in a given culture, fewer first cells than second cells. The “limited number” of first cells can be defined in absolute terms (e.g., less than about 100, 1,000, 10,000 or 100,000 cells) or in relative terms with respect to the number of second cells (e.g., about 1%, 2%, 5%, 10%, 15%, or 25% as many first cells as second cells). In any embodiment, the first cell and/or the second cell can be of a primary culture or an established cell line.

The term “about” is used herein to indicate that a value includes an inherent variation of error for the device or the method being employed to determine the value or plus-or-minus 10% of the stated value, whichever is greater. For example, about 100 cells is 90-110 cells.

In keeping with convention, we often refer herein to a first entity (e.g., a first cell) and a second entity (e.g., a second cell) to convey that the first and second entities are distinct from one another in some way (e.g., by including or excluding a virus). One of ordinary skill in the art will recognize that biological cells are rarely cultured alone, and it will be understood that where we refer to a first cell and/or a second cell, those cells can be and are in fact likely to be one of a plurality of cells, and the compositions and methods described herein apply to pluralities of cells. Accordingly, the invention features methods in which an individual cell is rendered virus free or in which the viral load is reduced as well as methods in which a population of cells is rendered virus free or in which the viral load is reduced (by, for example, eliminating at least one type of virus from some of the cells (but not others) or generally lowering the amount of the virus present in essentially all of the cells of the population).

In another aspect, the present invention features a virus-free cell or a population of cells (e.g., a virus-free cell line), or a cell or population of cells (e.g., a cell line) that carries a reduced viral load, made by a method described herein. The cell, prior to being subjected to such a method, may have been infected with a virus as described herein (e.g., a virus within the family Ascoviridae, Baculoviridae, Birnaviridae, Dicistroviridae, Iridoviridae, Metaviridae, Nodaviridae, Parvoviridae, Polydnaviridae, Poxviridae, Pseudoviridae, Reoviridae, or Tetraviridae). As noted, cells infected with other viruses, or any combination of viruses, can also be treated. Other viruses amenable to eradication by the present methods are described below. In one embodiment, the virus-free cell is of a cell line derived from Trichoplusia ni (e.g., of the cell line BTI-TN-5B1-4, HSCL-B (ATCC Accession No. PTA-5635), H5CL-F (ATCC Accession No. PTA-5636), BTI-TN-MG1 (ATCC Accession No. CRL-10860), or Hink's Trichoplusia ni (TN-368) cell line).

### BRIEF DESCRIPTION OF THE DRAWINGS

FIGS. 1A-1C are photomicrographs of cell cultures. Panel (A) shows 3-day old Manduca sexta (Ms) and Trichoplusia ni (T. ni) primary cultures from egg tissue before High Five™ cells (BTI-TN-5B1-4) were added. Panel (b) shows the fiber-like networks produced by the Ms and T. ni primary cultures after 14 days in culture. Panel (C) shows the distinct High Five™ cells (BTI-TN-5B1-4) among the primary cultures of the Ms and T. ni 10 days after the co-culture was initiated.

FIGS. 2A and 2B are photographs of agarose gels showing the RT-PCR products obtained from three flasks (F1, F2, and F3) of High Five™ cells (BTI-TN-5B1-4) co-cultured with primary Ms cells. “Hi5+Ms-PC” refers to High Five™ cells (BTI-TN-5B1-4) co-cultured with primary Ms cells. “Px” refers to the passage number. For example, P2 is a co-culture passaged twice. In FIG. 2A, “RNA1→” points to an amplified DNA segment of RNA1, one of the TNCL (Tn5 cell line) virus genomes. In FIG. 2B, “COI→” points to the amplified DNA segment of control RNA, COI.

FIG. 3 is a photograph of an agarose gel with the RT-PCR products of alphanodaviral RNA1 in the cell types and cultures indicated. When T. ni cells (primary cultures) were co-cultured with High Five™ cells (BTI-TN-5B1-4), there was no loss of alphanodavirus from the High Five™ cells (BTI-TN-5B1-4). Water (H₂O) and Sf9 cells (Sf9, an alphanodavirus-resistant cell line) were analyzed as negative controls, and High Five™ cells (BTI-TN-5B1-4) were analyzed as a positive (alphanodavirus-positive) control.

FIGS. 4A and 4B are photographs of agarose gels showing the results of co-culturing High Five™ cells (BTI-TN-5B1-4) and primary Manduca sexta cells in the presence of a barrier (a well insert) to prevent physical contact between the segregated cell types. In panel A, the agarose gel was loaded with RT-PCR products generated by amplifying alphanodavirus RNA1. In panel B, the agarose gel was loaded with RT-PCR products generated by amplifying the control RNA COI.

FIGS. 5A and 5B are photographs of agarose gels showing the presence or absence of alphanodavirus in different cell lines and High Five™ cell (BTI-TN-5B1-4) co-cultures. The gels show amplified RT-PCR products of TNCL viral RNA1 (Panel A), and of control RNA (CO1; a constitutively expressed cell line transcript) (Panel B).

FIG. 6 is a list of the ingredients in TNM-FH medium.

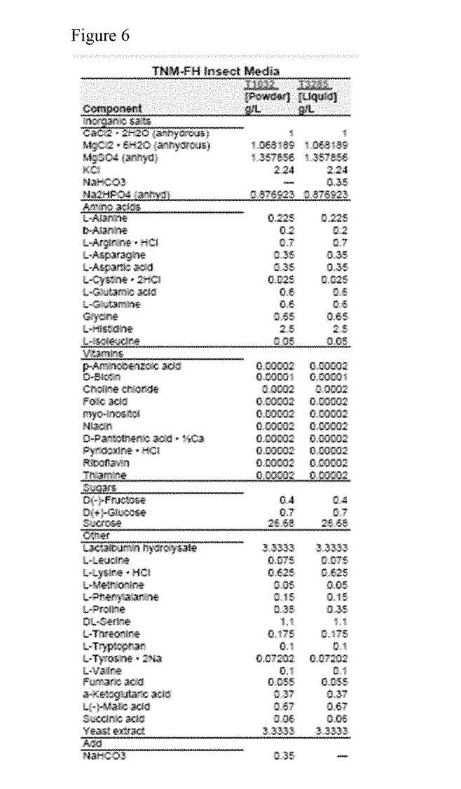

### DETAILED DESCRIPTION

A cell line established from embryonic tissue of Trichoplusia ni (cabbage looper; BTI-TN-5B1-4, ATC CRL 10859) is susceptible to various baculoviruses, including TnSNPV and AcMNPV, and has been used extensively as an expression system. It was also discovered that this cell line carries a virus of the genus Alphanodavirus as a persistent infection, and we have discovered and further developed various culture techniques for eliminating viruses from infected cells, or “curing” the culture of this viral infection. While there is no evidence that the alphanodavirus harms the cells or is detrimental to their use, it may generally be desirable to work with cells that are free of viral contamination in order to facilitate more convenient (clean) purification of recombinant products such as proteins and VLPs (virus-like particles). Accordingly, the methods of the invention encompass a process for eliminating alphanodavirus, or any one or more of many other viruses, from an infected cell line, and the resulting cells are also within the scope of the present invention.

The alphanodavirus-free cells can be used in any way, including any way the corresponding virus-infected cells can be used. Insect cells and baculovirus expression vectors have been used for many years and have become important in producing viral insecticides and expressing heterologous gene products of interest in the areas of biology, medicine, and agriculture to produce many heterologous proteins (see, e.g., Luckow and Summers, Virology 170:311-339, 1988). Accordingly, the present invention encompasses methods of producing a gene product by using, as the expression system, a population of cells or a cell line that has been treated as described herein to be virus free. Cell lines from Trichoplusia ni eggs have been established and infected (Rochford et al., In Vitro 20:823-825, 1984; and Granados et al., Virology 152:472-476, 1986), and a Trichoplusia ni embryonic cell line that is highly susceptible to numerous baculoviruses and efficiently supports replication of baculoviruses is described in U.S. Pat. No. 5,298,418. This cell line is available for use as described herein from the American Type Culture Collection (10801 University Boulevard, Manassas, Va. 20110 USA) under Accession No. ATC CRL 10859. The alphanodavirus-free cells produced by the present methods can be used to replicate baculoviruses (e.g., inoculated with baculoviruses AcMNPV and TnSNPV at an MOI of 5 and then incubated) and to produce recombinant proteins including antibodies, antitoxins, protein assemblies, antigens for vaccine therapy and any other therapeutic peptide or protein. As usual, the cells can be frozen in liquid nitrogen for safekeeping until use, and such stocks are within the scope of the present invention.

The methods described herein are designed to free a cell from viral infection, and they can be carried out in a series of steps that include co-culturing a first cell that is infected with the virus and a second cell that is not infected with the virus or susceptible to infection with the virus. The cells can be maintained in culture until the first cell is virus free. In case there is any doubt, and although we refer to a first cell and a second cell, it will be apparent to one of ordinary skill in the art that the methods can be practiced with populations of cells, either or both of which can be the cells of a clonal cell line. The present methods are not limited by any underlying mechanism; they may free a cell from viral infection by effectively eliminating a virus from a cell or they may foster, within a mixed population of cells, survival of non-infected cells and death of infected cells.

The first cell can be a cell from, or a cell line derived from, an invertebrate (e.g., an insect) or a vertebrate, including a mammal (e.g., a human). In some embodiments, the first cell is a cell of a cell line established from embryonic tissue of an insect, such as a moth (e.g., Trichoplusia ni) or is a cell included in a mixed population of cells (e.g., a heterogeneous population of insect cells). In some embodiments, the first cell is of the cell line designated BTI-TN-5B1-4, ATC CRL 10859. In other embodiments, the first cell is of the cell line HSCL-B (see U.S. Pat. No. 7,179,648, incorporated herein by reference) or HSCL-F (see U.S. Pat. No. 7,179,648, incorporated herein by reference), both of which were established from embryonic tissue of Trichoplusia ni. Cell lines established from embryonic tissue of Trichoplusia ni are susceptible to various baculoviruses, including TnSNPV and AcMNPV.

A High Five™ cell line (BTI-TN-5B1-4) of Trichoplusia ni from which alphanodavirus has been removed by the methods of the invention was deposited under the terms of the Budapest Treaty on the International Recognition of the Deposit of Microorganisms for the Purposes of Patent Procedure at the American Type Culture Collection (ATCC), 10801 University Boulevard, Manassas, Va. 20110-2209 (USA) under deposit Accession No. ATCC PTA-120815 on Jan. 7, 2014. All restrictions imposed by the depositor on the availability to the public of the deposited material will be irrevocably removed upon the granting of a patent.

In some embodiments, the virus (or one of the viruses) within the first cell is a double-stranded DNA (dsDNA) virus, a single-stranded DNA (ssDNA) virus, a retrovirus containing a single-stranded RNA genome (ssRNA-RT), a double-stranded RNA (dsRNA) virus, or a positive or negative single-stranded RNA (ssRNA+ or ssRNA−, respectively) virus. Accordingly, the present methods are useful in removing either RNA or DNA viruses from infected cells. Any cell infected with such a virus can be treated as the “first cell” in the present methods. Examples of dsDNA viruses are those within the family Herpesviridae, Adenoviridae, Asfarviridae, Nimaviridae, Papillomaviridae, Polyomaviridae, or Poxviridae. Examples of ssDNA viruses are those within the family Circoviridae, Parvoiridae, Hepadnaviridae, or Metaviridae. Viruses within the family Retroviridae are ssRNA-RT viruses. Where the virus is a negative strand ssRNA virus, it may be one within the family Bornaviridae, Filoviridai, Paramyxoviridae, Rhabdoviridae, Arenaviridae, Bunyaviridae, Orthomyxoviridae, or Deltavirus. Where the virus is a positive strand ssRNA virus, it may be one within the family Arteriviridae, Coronaviridae, Picornaviridae, Tymoviridai, Astroviridae, Calciviridae, Flaviviridae, Herpeviridae, Nodaviridae, Rhabdoviridae, Tetraviridae, or Togaviridae. Viruses within the families Nimaviridae, Poxviridae, Parvoviridae, Metaviridae, Birnaviridae, Reoviridae, Rhabdoviridae, Bunyaviridae, Orthomyxoviridae, Picornaviridae, Dicistroviridae, Flaviviridae, Nodaviridae, Rhabdoviridae, Tetraviridae, and Togaviridae can infect invertebrate hosts or both vertebrate and invertebrate hosts. Accordingly, the methods aimed at eradicating a virus within these families may be carried out with cells or cell lines derived from either invertebrates or vertebrates.

Ascoviruses are double-stranded DNA viruses that infect primarily invertebrates (e.g., invertebrates within the order Lepidoptera). The family extends to a single genus (Ascovirus), within which there are currently six known species. Ascoviruses may have evolved from iridoviruses, and viruses within the family Iridoviridae can also be eliminated by the present methods. In contrast, viruses within the family Nodaviridae are RNA viruses. Within Nodaviridae, the genome is linear, positive sense, bipartite single-stranded RNA. Thus, within one embodiment, In some embodiments, the virus is an RNA virus. In some embodiments, the virus is a single stranded RNA (ssRNA) virus. In certain embodiments, the virus is a positive sense (+) ssRNA virus. In some embodiments, the ssRNA (+) virus is within the family Nodaviridae. In some embodiments, the ssRNA (+) virus is within the genus Alphanodavirus. In certain embodiments, the ssRNA (+) virus within the genus Alphanodavirus is Nodamura Virus, Flock House Virus (FHV), Black Beetle Virus, Boolarra Virus, Pariacoto Virus, Macrobrachium rosenbergii nodavirus, Penaeus vannamei nodavirus, or Tn5 Cell Line Virus (TNCLV; a Tn-5-derived nodavirus).

Viruses within the family Baculoviridae are divided between the genera alpha-, beta-, gamma- and delta-baculovirus. Many invertebrate species can be infected by baculoviruses, and the present methods are useful in eradicating such infections from infected cells in culture.

Viruses within the family Rhabdoviridae (i.e., Rhabdoviruses) that can be eliminated or reduced according to the present methods can be within the genus Lyssavirus, Novirhabdovirus, Ephemerovirus, Perhabdovirus, Tibrovirus, Nucleorhabdovirus, Tupavirus, Vesiculovirus, Sprivivirus, Cytorhabdovirus, or Sigmavirus. In certain embodiments, the virus is within the genus Cytorhabdovirus and can be Rf-Rhabdovirus.

Rhabdoviruses carry their genetic material in the form of negative-sense single-stranded RNA (i.e., (−) ssRNA). They typically carry genes for five proteins: large protein (L), glycoprotein (G), nucleoprotein (N), phosphoprotein (P), and matrix protein (M).

In keeping with the definition provided above, a cell is considered virus-free when it is tested by a currently available methodology and found to lack any detectable level of one or more specific viruses. For example, if one knows that cells in a cell culture or cells of a cell line are infected with an Alphanodavirus, and one wishes to eradicate the Alphanodavirus from the cell culture or cell line, the cells are virus-free when the Alphanodavirus levels fall below a detectable level. We may also use the term virus-free to describe the status of a cell having a particularly identified virus. For example, a cell that has been freed of Alphanodavirus may be described as virus-free with respect to that virus (i.e., the cell may include, but does not necessarily include, other viruses). Similarly, a cell may be free of TNCLV but continue to include other viruses; a cell may be free of a virus within the Rhabdoviridae family but include viruses from other families; and so forth. In some embodiments, the second cell is from a species other than the first cell. In some embodiments, the second cell is alphanodavirus-free. In some embodiments, the second cell is a cell line of a primary culture of Manduca sexta cells; of a Manduca sexta cell line; or of the Sf9 cell line. In case of any doubt, a given cell type may be infected with a virus and used as the “first” cell in the present methods in some instances and may be virus-free and used as the “second” cell in the present methods in other instances.

### EXAMPLES

### Example 1: Co-Culturing High Five™ Cells (BTI-TN-5B1-4) and Manduca sexta Primary Cultures Cured the High Five™ Cells (BTI-TN-5B1-4) from Infection with Alphanodavirus

We made primary cultures of Manduca sexta eggs from 1-3 day-old eggs obtained from Dr. Gary Blissard's laboratory at the Boyce Thompson Institute. The “Ms” cell line was designated MRRL-CH1 and had a passage number of 31. We made primary cultures of Trichoplusia ni from T. ni eggs obtained from Dr. Ping Wang's laboratory, Cornell University. To establish primary cultures from the insect eggs, we collected 300-500 eggs and disinfected them with Clorox™ (5% bleach) for one minute. We then rinsed the eggs (×3) in autoclaved water and, working in a tissue culture hood, transferred them into a cell strainer that was submerged in a well of a six-well plate containing 70% ethanol. After five-minutes, we rinsed the eggs (2×) with autoclaved water and then with 5 ml of TNM-FH medium with FBS (Hyclone, Catalog No. SH30071.03) supplemented with antibiotics (×3). We then crushed the eggs in the cell strainer with the handle of a cell scraper and pushed the egg tissue through the membrane of the strainer into a well of a new six-well plate containing 5 ml of the TNM-FH medium. The suspension containing the egg tissue was diluted with fresh medium up to a volume of 30 ml. We transferred 3 ml into each of six T25 flasks (18 ml total), and we transferred 0.5 ml into each of the wells of two 12-well plates (12 ml total). These primary cultures were incubated at 27° C. for four days. We transferred the supernatant from the primary cultures to a new set of T25 flasks before replacing the medium with fresh medium. All flasks were labeled and dated.

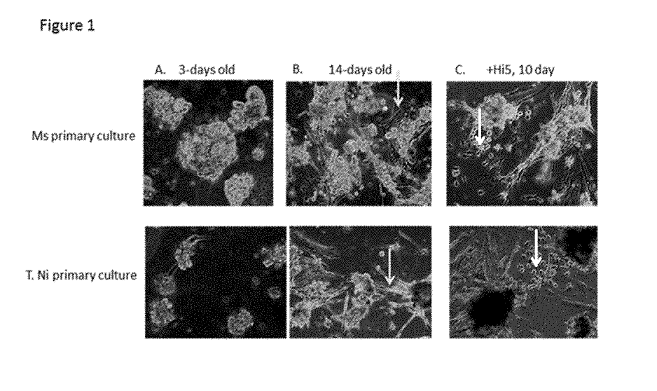

After plating, the egg tissues slowly adhered to the bottoms of the flasks and wells. The density of the egg tissues in a flask can affect the outcome when the tissue is later co-cultured with High Five™ cells (BTI-TN-5B1-4). We achieved good results by leaving the egg tissues undisturbed for the first three days after plating. On the fourth day, when most of the egg tissues had adhered to the tissue culture vessels (FIG. 1A), we added High Five™ cells (BTI-TN-5B1-4) at very low density, from our low passage stock (cells passaged only 80-90 times), to the primary cultures. Generally, we added less than 10 High Five™ cells (BTI-TN-5B1-4) per T25 flask. We could accomplish this minimal transfer of High Five™ cells (BTI-TN-5B1-4) by gently dipping a nearly empty pipette tip that had been used to suspend the High Five™ cell (BTI-TN-5B1-4) culture into the primary culture. The High Five™ cells (BTI-TN-5B1-4) added to the primary culture were designated as passage zero (P0). The culture medium was changed the next day. Cell growth was monitored every 2-3 days, and the medium was changed weekly. The High Five™ cells (BTI-TN-5B1-4) grew very slowly during the first week, but once a colony was established, the cells grew more rapidly among the explanted egg tissues. The latter were growing at the same time and formed fiber-like networks in most of the cultures (FIG. 1B). The appearance of the High Five™ cell (BTI-TN-5B1-4) colonies was distinct from the appearance of the egg tissues (FIG. 1C). When the High Five™ cells (BTI-TN-5B1-4) formed large colonies among the egg tissues and started to grow on top of each other (in 2-3 weeks), we knocked the cells off the flask and resuspended them. The suspension culture containing the High Five™ cells (BTI-TN-5B1-4) was used to spike a new T25 flask containing the same type of primary culture. Once transferred, the High Five™ cells (BTI-TN-5B1-4) were designated as first passage (P1). This procedure was repeated until the passage number reached P4 or higher. Cells from each passage were saved and grown up for RNA isolation and alphanodavirus analysis. Three replicates were performed in each experiment.

Total cellular RNA was isolated from the cells at different passages and assayed for alphanodavirus by RT-PCR. The RNA was isolated with TRIzol® reagent using the manufacturer's protocol (Life Technologies). To prepare each sample, cells were harvested from a T25 flask. The RNA was dissolved in DEPC-treated water and kept at −70° C. To detect alphanodavirus, we used a one-step RT-PCR method as previously described (Hashimoto et al., BMC Biotechnol. 10:50, 2010; Li et al., J. Virol. 81:10890-10896, 2007; and Shan et al., Virol. Sin. 26:297-305, 2011). The primer set for alphanodavirus RNA1 included Noda-R1-2368F (5′-TGTACCGATGCGCTTACTCCGTTGATATCGG-3′ (SEQ ID NO:1)) and Noda-R1-2933R (5′-CCACGCTGGGTTTCTCCAGCAGTGATGTTACC-3′ (SEQ ID NO:2). The end product of the RT-PCR is a 565 bp DNA fragment. To verify the qualities of RNA samples in each RT-PCR reaction, we used the mitochondrial gene CO1 (cytochrome C oxidase subunit 1) as the internal control. The primer set for CO1 was LCO1490 (5′-GGTCAACAAATCATAAAGATATTGG-3′ (SEQ ID NO:3)) and HCO2198 (5′-TAAATCTCAGGGTGACCAAAAAATCA-3′; SEQ ID NO:4)), which produced a DNA fragment 658 bp (Folmer et al., Mol. Mar. Biol. Biotechnol. 3:294-299, 1994; Lukhtanov et al., Mol. Ecol. Resour. 9:1302-1310, 2009; and Hanhimoto et al., BMC Biotechnol. 12:12, 2012). One-step RT-PCR was performed using the Invitrogen SuperScript III One-Step RT-PCR System with Platinum Taq DNA polymerase under the following conditions: 45° C. for 30 minutes; 94° C. for two minutes; 40 repeats of the cycle 94° C. for 15 seconds, 55° C. for 30 seconds, and 72° C. for 45 seconds; and 72° C. for 10 minutes. The PCR products were analyzed on 1.4% agarose gels.

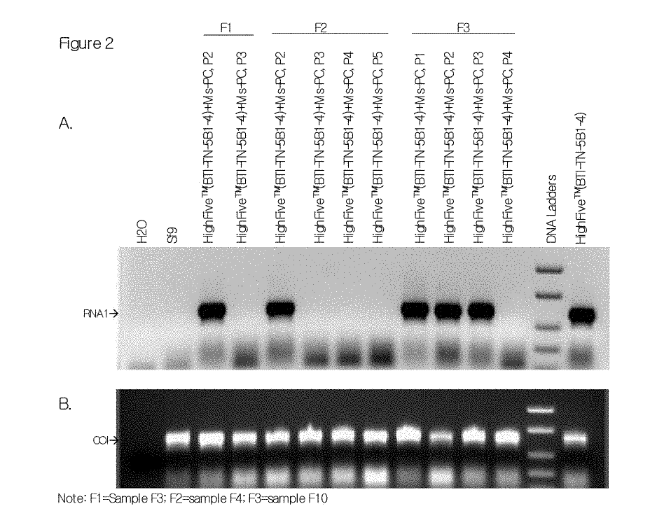

The alphanodavirus (Tn5 cell line virus or TNCL virus) was removed from the High Five™ cells (BTI-TN-5B1-4) after 3 or 4 passages in co-culture with the Manduca sexta primary cultures described above. FIG. 2A shows the RT-PCR products obtained from three flasks (F1, F2, and F3) of co-cultured High Five™ cells (BTI-TN-5B1-4) and Manduca sexta primary cultures (“Hi5+Ms-PC”). In F1, the amplified TNCL viral nucleic acid is clearly visible at passage 2 (P2) but not passage 3 (P3). In F2, TNCL viral nucleic acid is clearly visible at P2 but not at P3, P4, or P5. In F3, TNCL viral nucleic acid is clearly visible at P1, P2, and P3 but not at P4. The uniform detection of the mitochondrial gene CO1 (FIG. 2B) rules out the possibility that the quality or quantity of the nucleic acids in the samples was responsible for the result.

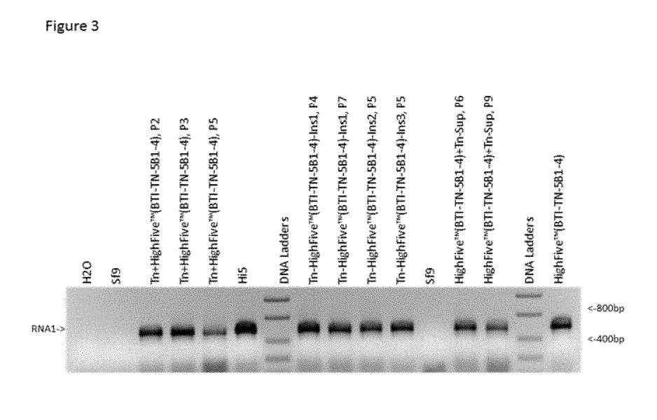

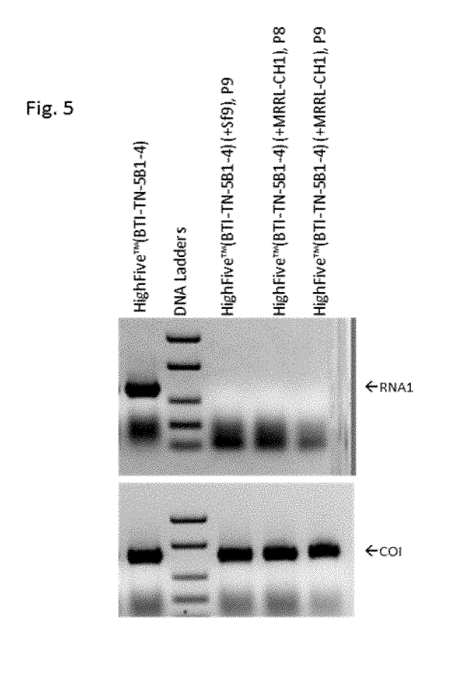

To determine whether the primary culture of T. ni cells had the same effect on alphanodavirus, we performed the same analysis on High Five™ cells (BTI-TN-5B1-4) that were co-cultured with T. ni primary cultures from eggs. In that instance, there was no loss of alphanodavirus from the High Five™ cells (BTI-TN-5B1-4) (see FIG. 3) even after seven passages. We tested the T. ni eggs for the presence of alphanodavirus and the results were negative (FIG. 5, lane 2). The results presented herein suggest Manduca sexta, but not T. ni primary cultures can cure High Five™ cells (BTI-TN-5B1-4) infected by alphanodavirus.

### Example 2: Culturing High Five™ Cells (BTI-TN-5B1-4) and Manduca sexta Primary Cultures in Separate Compartments Cured the High Five™ Cells (BTI-TN-5B1-4) from Infection with Alphanodavirus

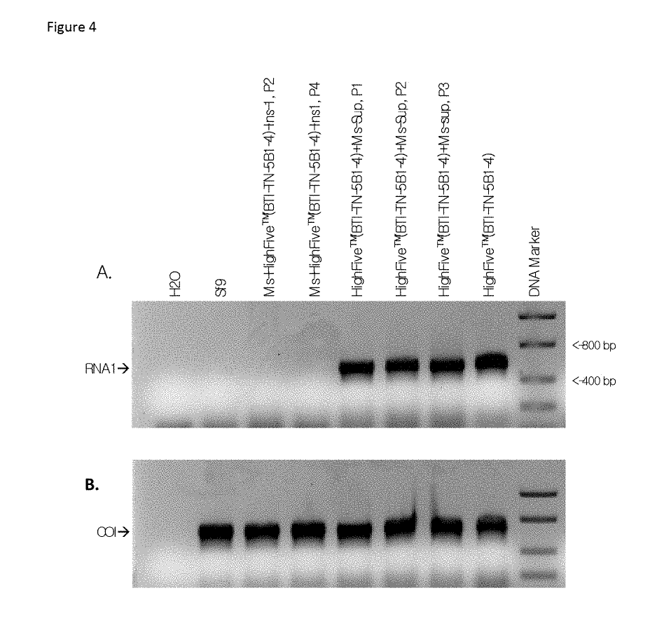

To determine whether the same phenomenon we observed above could be achieved by co-culturing High Five™ cells (BTI-TN-5B1-4) with primary cultures in separate compartments, we cultured M. sexta egg tissues as described above and High Five™ cells (BTI-TN-5B1-4) within an insert that allowed both cell types exposure to the same tissue culture medium. More specifically, four days after the primary M. sexta cultures were established, we placed an insert (a TC Insert for 12-well plates; ThinCert®, Greiner Bio-One, Catalog No. 665641), into the culture dish. High Five™ cells (BTI-TN-5B1-4) (<10 cells) were added within the insert in 0.7 mL of medium with antibiotics (a mixture of penicillin, streptomycin, and amphotericin B, Invitrogen, 100×, Catalog No. 15240096; to a final concentration of 1×). If there were too many cells (>10) within an insert, we diluted them in the next day or two. The insert was transferred into a new well weekly, with or without passaging. When the High Five™ cells (BTI-TN-5B1-4) became 40-50% confluent, we detached them by repetitive pipetting. The cell suspension was withdrawn and fresh medium was added to the residual High Five™ cells (BTI-TN-5B1-4) remaining on the insert membrane. Those remaining cells in the insert continued to grow after the insert was placed in a new well. After two passages, a few of the High Five™ cells (BTI-TN-5B1-4) (<10) were transferred into a new insert, and the rest of the cell suspension was transferred into a T25 flask and saved for total RNA isolation. Three inserts were used for each of the co-culturing experiments. As shown in FIG. 4A, High Five™ cells (BTI-TN-5B1-4) co-cultured with M. sexta primary cultures under these conditions were cured from the TNCL virus infection after only two passages (see lanes 3 and 4). CO1 was again amplified as a control for the quantity and quality of the RNA tested (FIG. 4B).

As we expected, under the same conditions, T. ni primary cultures failed to cure High Five™ cells (BTI-TN-5B1-4) (FIG. 3, lane 8 to lane 11) even after 7 passages. We also tested the T. ni egg tissues after co-culturing with High Five™ cells (BTI-TN-5B1-4) as described here. These egg tissues appeared to be infected by alphanodavirus as a result of growing in the same medium as the High Five™ cells (BTI-TN-5B1-4) (data not shown).

### Example 3: Culturing High Five™ Cells (BTI-TN-5B1-4) in Conditioned Medium (Supernatant) Previously Used to Culture Primary Cells, Failed to Cure the High Five™ Cells (BTI-TN-5B1-4) from Infection with Alphanodavirus

We cultured the egg tissues as described above in TNM-FH medium containing antibiotics. During the first three weeks after initiation, the spent media from these primary cultures were frozen, then pooled together, and filtered through a 0.2 μm filter. We then cultured High Five™ cells (BTI-TN-5B1-4) in the filtered, spent medium at very low cell density (<10 cells per flask). The cells grew very slowly under these conditions, so no medium change was necessary until the cells started to form small colonies (about 2-3 weeks). When the cells reached about 50% confluence, we resuspended and passaged them into new T25 flasks containing the same spent medium at very low density (<10 cells/flask). Cells at each passage were saved for total RNA isolation and alphanodavirus analysis. As shown in FIGS. 3 and 4, it is evident that High Five™ cells (BTI-TN-5B1-4) were not cured when grown in the spent medium of either M. sexta or T. ni primary cell cultures. Given the success we have had in eliminating alphanodavirus from infected cells when those cells are co-cultured with, but separated from, alphanodavirus-resistant cells in a co-culture (i.e., when the two cell types are separated by a well insert), we suspect that there is a factor in the design of this experiment that is preventing us from seeing a similar, curative outcome. We are evaluating our experimental design, and more work will have to be done before we can conclude that transfer of a supernatant, spent culture medium, or a factor or factors therein is ineffective in curing alphanodavirus-infected cells.

### Example 4: Culturing High Five™ Cells (BTI-TN-5B1-4) with Established Cell Lines that are not Susceptible to Alphanodavirus, Also Cured the High Five™ Cells from Infection with the TNCLV Alphanodavirus

Because M. sexta primary cultures but not spent medium could cure High Five™ cells (BTI-TN-5B1-4), we hypothesized that the curing function of M. sexta primary culture could be due to its resistance to TNCL viral infection. As noted, tests on RNA obtained from M. sexta primary cultures failed to show any TNCL virus (FIG. 5, lane 3). We used a cloned M. sexta cell line, MRRL-CH, to test the susceptibility of M. sexta cultures to TNCL virus, along with three control cell lines—Sf9, Tnao38, and Tnms42. Sf9 cells (an insect cell line derived from the parental Spodoptera frugiperda cell line IPLB-Sf-21-AE) are not susceptible to TNCL virus while Tnao38 and Tnms42, two clonal lines of High Five™ cells (BTI-TN-5B1-4), are known to be susceptible (Hashimoto et al., BMC Biotechnol. 10:50, 2010). The supernatant of High Five™ cell (BTI-TN-5B1-4) cultures also contains readily detectable levels of TNCL virus (Li et al., J. Virol. 81:10890-10896, 2007). Therefore, we used High Five™ cell (BTI-TN-5B1-4) culture medium to treat these four cell lines. Briefly, High Five™ cells (BTI-TN-5B1-4) were cultured in a T75 flask containing 12 ml of TNM-FH medium until reaching 70-90% confluency (in about 3 days). The medium was collected and filtered through a 0.2 μm filter. The cell lines Sf9, MRRL-CH1, Tnao38 and Tnms42 were first plated in T25 flasks a day before the infection. The cell densities were controlled at about 60-75% confluency. After replacing the cell medium with 2 ml of the filtered High Five™ cell (BTI-TN-5B1-4) medium, the cells were incubated at 27° C. for 1 hour. We then removed the medium and rinsed the cell surfaces with fresh TNM-FH medium (×3) to remove much of the free alphanodavirus particles. The cells were allowed to grow in a 27° C. incubator until reaching 75-90% confluency. The cells were sub-cultured through at least six passages before being subjected to total RNA isolation and alphanodavirus assays as described above. This allowed for any free TNCL virus particles carried over from the treatment to be removed. Total RNA was isolated from each of the cell lines and subjected to alphanodavirus analysis. As shown in FIG. 5, the four cell lines were negative for TNCL virus before the treatment, and both MRRL-CH1 and Sf9 cells remained negative after the treatment (lanes 8 and 9). The two T. ni cell lines (Tnao38 and Tnms42) were positive for the presence of TNCL virus after treatment (lanes 10 and 11) as expected. These results confirmed our hypothesis that Ms cells are indeed not susceptible to TNCL virus.

Based on the susceptibility test described above, we co-cultured High Five™ cells (BTI-TN-5B1-4) with the two non-susceptible cells lines (Sf9 and MRRL-CH1) to determine whether they could also cure High Five™ cells (BTI-TN-5B1-4). The Sf9 and MRRL-CH1 cells were plated separately in a 6-well plate at cell densities of 3×10⁵ and 5×10⁵ cells per well, respectively. High Five™ cells (BTI-TN-5B1-4) were spiked into these two wells at very low density (<10 cells/well). When the mixed cell density reached about 80-90% confluence (in about 5 days), the cells were passaged (P0 to P1) into a new well with 10-fold dilution. Because the doubling time of High Five™ cells (BTI-TN-5B1-4) is shorter than the doubling time of the other two cells lines (21 hours for High Five™ cells (BTI-TN-5B1-4), 24 hours for Sf9; and more than 30 hours for MRRL-CH1), High Five™ cells (BTI-TN-5B1-4) would quickly outgrow the other cells at passage 1. We only transferred 50 μl of the cells from passage 1 into a new well containing the other cells (Sf9 or MRRL-CH1) at a cell density of 5×10⁵ cells/well for the MRRL-CH1 cells and 3×10⁵ cells/well for the Sf9 cells. We repeated this procedure until the passage number reached P6 or higher. Cells at P6 or higher were saved and grown up for total RNA isolation and alphanodavirus detection by RT-PCR as described above.

The distinctive morphologies of the Sf9 and MRRL-CH1 cells made it easy to monitor the progress of High Five™ cell (BTI-TN-5B1-4) growth during the period of co-culture. When the passage number reached P8, we allowed the mixed cells to grow for an additional two weeks without the addition of any more cells from either the Sf9 or MRRL-CH1 cell lines. This allowed the High Five™ cells (BTI-TN-5B1-4) to take over the culture, becoming the majority cell type. In fact, by the end of the two week period, it was difficult to find any cells other than High Five™ cells (BTI-TN-5B1-4) by visual inspection. The assay results are shown in FIG. 5. We found that High Five™ cells (BTI-TN-5B1-4) could be cured of alphanodavirus infection by co-culturing them with both the Sf9 and MRRL-CH1 cells, suggesting that High Five™ cells (BTI-TN-5B1-4) could be cured of alphanodavirus infection by co-culturing them with cells that are not susceptible to this virus. To confirm any impressions formed by visual inspection, one can assess a marker expressed by the originally cultured cell type(s), and the present methods can include such a step. For example, one could expose the culture to antibodies that specifically bind an expressed antigen or amplify a known gene sequence by PCR in order to help confirm the identities of the cultured cells.

In the future, we are contemplating cloning the cells that have been cured (previously, the High Five™ cells (BTI-TN-5B1-4)) and sequencing the cell line to confirm a clonal population.

### Prophetic Example 5: Co-Culturing Sf9 Cells (e.g., BTI-TN-5B1-4) with Manduca sexta Primary Cultures to Cure the Sf9 Cells from Infection with Sf-Rhabdovirus

Recently, it was discovered that Sf9 cells obtained from two commercial sources were contaminated with a rhabdovirus known as Sf-rhabdovirus (Ma et al., J. Virol. 88:6576-6585, 2014). To determine whether results similar to those we observed above could be achieved by co-culturing Sf9 cells with Manduca sexta primary cultures, one could co-culture M. sexta egg tissue and SD cells (e.g., within an insert that allows both cell types exposure to the same tissue culture medium).

One could take primary cultures of Manduca sexta eggs from 1-3 day-old eggs, such as described above in Example 1. For example, establish primary cultures from the insect eggs, one could collect about 300-500 eggs and disinfect them with Clorox™ (5% bleach) for one minute. One could then rinse the eggs (×3) in autoclaved water and, working in a tissue culture hood, transfer them into a cell strainer submerged in a well of a six-well plate containing 70% ethanol. After five minutes, the eggs could be rinsed (2×) with autoclaved water and then with 5 ml of TNM-FH medium with FBS (Hyclone, Catalog No. SH30071.03) supplemented with antibiotics (×3). The eggs would then be crushed in the cell strainer with the handle of a cell scraper and pushed through the membrane of the strainer into a well of a new six-well plate containing 5 ml of the TNM-FH medium. The suspension containing the egg tissue would be diluted with fresh medium up to a volume of 30 ml. One would then transfer 3 ml into each of six T25 flasks (18 ml total), and transfer 0.5 ml into each of the wells of two 12-well plates (12 ml total). These primary cultures would be incubated at 27° C. for four days. One would then transfer the supernatant from the primary cultures to a new set of T25 flasks before replacing the medium with fresh medium.

After plating, the egg tissues will slowly adhere to the bottoms of the flasks and wells. On about the fourth day, when most of the egg tissues will have adhered to the tissue culture vessels, one would add Sf9 cells at very low density to the primary cultures. Generally, one would add less than about 10 Sf9 cells per T25 flask. One could accomplish this minimal transfer of Sf9 cells by gently dipping a nearly empty pipette tip that had been used to suspend the Sf9 cell culture into the primary culture. The Sf9 cells added to the primary culture would be designated as passage zero (P0). The culture medium would be changed the next day. Cell growth would be monitored every 2-3 days, and the medium changed weekly. The appearance of the Sf9 cell colonies would be distinct from the appearance of the egg tissues. When the Sf9 cells formed large colonies among the egg tissues and started to grow on top of each other (in 2-3 weeks), one would knock the cells off the flask and re-suspended them. The suspension culture containing the Sf9 cells would be used to spike a new T25 flask containing the same type of primary culture. Once transferred, the Sf9 cells would be designated as first passage (P1). This procedure would be repeated until the passage number reached P4 or higher. Cells from each passage would be saved and grown up for RNA or DNA isolation and viral analysis.

Total cellular RNA would be isolated from the cells at different passages and assayed for Sf-rhabdovirus by RT-PCR. The RNA could be isolated with TRIzol® reagent using the manufacturer's protocol (Life Technologies). To prepare each sample, cells would be harvested from a T25 flask. The RNA would be dissolved in DEPC-treated water and kept at −70° C. To detect Sf-rhabdovirus, one could use a one-step RT-PCR method using virus-specific primers and conditions optimal for RT-PCR (e.g., the primers described in WO 2011/072276 at Table 2).

### Prophetic Example 6: Culturing SF9 Cells with Manduca sexta Primary Cultures in Separate Compartments could be Used to Cure Sf9 Cells from Infection with Sf-Rhabdovirus

To assess the effect of co-culturing Sf9 cells with primary cultures in separate compartments, one could co-culture M. sexta egg tissues as described above and Sf9 cells with or within an insert that allows both cell types exposure to the same tissue culture medium. More specifically, four days after the primary M. sexta cultures are established, one could place an insert (a TC Insert for 12-well plates; ThinCert®, Greiner Bio-One, Catalog No. 665641), into the culture dish. Sf9 cells (<10 cells) could be added within the insert in 0.7 mL of medium with antibiotics (a mixture of penicillin, streptomycin, and amphotericin B (Invitrogen, 100x, Catalog No. 15240096;) to a final concentration of lx). If there were too many cells (>10) within an insert, one could dilute them in the next day or two. The insert would be transferred into a new well weekly, with or without passaging. When the Sf9 cells become approximately 40-50% confluent, one could then detach them by repetitive pipetting. The cell suspension would be withdrawn and fresh medium would be added to the residual Sf9 cells remaining on the insert membrane. Those remaining cells in the insert could continue to grow after the insert is placed in a new well. After two passages, a few of the Sf9 cells (<10) would be transferred into a new insert, and the rest of the cell suspension would be transferred into a T25 flask and saved for total RNA isolation. This could be continued for a number of passages until the Sf9 cells are cured of the Sf-rhabdovirus.

What is claimed is:

1. A method of making a virus-free cell, the method comprising co-culturing a first cell that is infected with a virus and a second cell that is not infected with the virus, wherein the cells are maintained in culture until the first cell is free of the virus.

2. The method of claim 1, wherein the virus is within the family Ascoviridae, Baculoviridae, Birnaviridae, Dicistroviridae, Iridoviridae, Metaviridae, Nodaviridae, Parvoviridae, Polydnaviridae, Poxviridae, Pseudoviridae, Rhabdoviridae, Reoviridae, or Tetraviridae.

3. The method of claim 1, wherein the virus is an RNA virus.

4. The method of claim 3, wherein the RNA virus is a single stranded RNA (ssRNA) virus.

5. The method of claim 4, wherein the ssRNA virus is a positive sense (+) ssRNA virus.

6. The method of claim 5, wherein the (+) ssRNA virus is within the family Nodaviridae.

7. The method of claim 6, wherein the (+) ssRNA virus is within the genus Alphanodavirus.

8. The method of claim 7, wherein the (+) ssRNA virus within the genus Alphanodavirus is Nodamura Virus, Flock House Virus (FHV), Black Beetle Virus, Boolarra Virus, TNCL Virus or Pariacoto Virus.

9. The method of claim 1, wherein co-culturing comprises placing the first and second cells in the same tissue culture vessel without any barrier to impede contact between the first and second cells or wherein co-culturing comprises placing the first and second cells in the same tissue culture vessel with a barrier that impedes contact between the first and second cells but not the medium in which they are grown.

10. (canceled)

11. The method of claim 1, wherein the first cell and the second cell are (a) different species within a genus; (b) of different genera; or (c) insect cells.

12.-13. (canceled)

14. The method of claim 1, wherein the first cell is of a cell line derived from Trichoplusia ni.

15. The method of claim 14, wherein the first cell is of the cell line BTI-TN-5B1-4.

16. The method of claim 1, wherein the second cell is of a primary culture of Manduca sexta cells; of an established Manduca sexta cell line; or of the Sf9 cell line.

17. The method of claim 1, wherein the second cell is cultured alone prior to the addition of the first cell.

18. (canceled)

19. A cell made by the method of claim 1.

20. (canceled)

21. The virus-free cell of claim 19, wherein the virus-free cell is of a cell line derived from Trichoplusia ni.

22. The virus-free cell of claim 19, wherein the virus-free cell is of the cell line High Five™ (BTI-TN-5B1-4), the cell line H5CL-B (ATCC Accession number PTA-5635), H5CL-F (ATCC Accession number PTA-5636), BTI-TN-MG1 (ATCC Accession number CRL-I0860), or Hink's Trichoplusia ni (TN-368) cell line.

23. (canceled)

24. A cell of the cell line having ATCC Accession No. PTA-120815.

25. A method of making a virus-free cell or reducing the viral load in a cell, the method comprising culturing a first cell that is infected with the virus in a culture medium in which a second cell that is not infected with the virus has been cultured, wherein the culture is maintained for a time sufficient for the first cell to become virus-free or to acquire a reduced viral load.

26. A virus-free cell made by the method of claim 25.
